(19) United States
(12) Patent Application Publication
(10) Pub. No.: US 2026/0325578 A1
(43) Pub. Date: Oct. 22, 2026

(54) KIT FOR PREPARING BIOACTIVE COMPOSITION CONTAINING NON-CROSSLINKED HYALURONIC ACID AND PLATELET-RICH PLASMA

(71) Applicants: Chia-Hua HSIEH, Taoyuan City (TW); Infelite Biomed Inc., Taichung City (TW)

(72) Inventor: Chia-Hua HSIEH, Taoyuan City (TW)

(21) Appl. No.: 19/648,553

(22) Filed: Apr. 15, 2026

(30) Foreign Application Priority Data
Apr. 16, 2025 (TW) 114114406

(51) Int. Cl. A61K 35/16; A61K 9/08; A61K 31/728; A61K 35/19

(52) U.S. Cl. CPC A61K 35/16; A61K 9/08; A61K 31/728; A61K 35/19

(57) ABSTRACT

A kit for preparing a bioactive composition containing non-crosslinked hyaluronic acid and platelet-rich plasma includes a surface-modified centrifuge tube having an inner surface, a modified hyaluronic acid solution contained in the surface-modified centrifuge tube and including the non-crosslinked hyaluronic acid and a solvent, and a modified separation gel contained in the surface-modified centrifuge tube. A zeta potential of the inner surface is not greater than −25 mV. A pH value of the modified hyaluronic acid solution ranges from 7.0 to 7.8, and a zeta potential of the non-crosslinked hyaluronic acid is not greater than −40 mV. The modified hyaluronic acid solution is disposed at an inner bottom of the surface modified centrifuge tube. A zeta potential of the modified separation gel is not greater than −25 mV. The modified separation gel is disposed above the modified hyaluronic acid solution.

### CROSS-REFERENCE TO RELATED APPLICATION

This application claims priority to Taiwanese Invention patent application No. 114114406, filed on Apr. 16, 2025, the entire disclosure of which is incorporated by reference herein.

### FIELD

The disclosure relates to a kit for preparing a bioactive composition containing non-crosslinked hyaluronic acid and platelet-rich plasma.

### BACKGROUND

Platelet-rich plasma (PRP) is rich in various growth factors, such as platelet-derived growth factor (PDGF), epidermal growth factor (EGF), and transforming growth factor-beta 1 (TGF-β1), which are beneficial for promoting tissue repair, stimulating cell proliferation, and accelerating healing at sites of injury. Therefore, PRP has been widely applied in fields such as regenerative therapy (e.g., prolotherapy), plastic surgery, sports medicine, and the treatment of degenerative joint diseases.

However, the growth factor concentration in PRP that is prepared by a conventional PRP preparation kit or device is still insufficient. In order to increase the growth factor concentration in PRP for achieving a desired therapeutic effect, an additional processing step such as performing cryoactivation (which requires a treatment time of at least 8 hours to 12 hours) or addition of an activator (which requires a treatment time of at least 30 minutes to 60 minutes) is typically necessary. Such additional processing steps are not only time-consuming but also may impair platelet activity, which compromises both real-time applicability of PRP and ease of operation for preparation of the same.

In view of the aforesaid, there is a need to develop a PRP preparation kit or device that is capable of obtaining PRP with improved growth factor concentration therein.

### SUMMARY

Therefore, an object of the disclosure is to provide a kit for preparing a bioactive composition containing non-crosslinked hyaluronic acid and platelet-rich plasma, which can alleviate at least one of the drawbacks of the prior art.

The kit includes a surface-modified centrifuge tube, a modified hyaluronic acid solution, and a modified separation gel.

The surface-modified centrifuge tube has an inner surface, and a zeta potential of the inner surface is not greater than −25 mV. The modified hyaluronic acid solution is contained in the surface-modified centrifuge tube, and includes the non-crosslinked hyaluronic acid and a solvent. A pH value of the modified hyaluronic acid solution ranges from 7.0 to 7.8, and a zeta potential of the non-crosslinked hyaluronic acid is not greater than −40 mV. The modified hyaluronic acid solution is disposed at an inner bottom of the surface-modified centrifuge tube. The modified separation gel is contained in the surface-modified centrifuge tube. A zeta potential of the modified separation gel is not greater than −25 mV. The modified separation gel is disposed above the modified hyaluronic acid solution.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a schematic view illustrating an example of a kit for preparing a bioactive composition containing non-crosslinked hyaluronic acid and platelet-rich plasma according to the present disclosure.

FIG. 2 is a schematic diagram illustrating a method for preparing the bioactive composition using the kit according to the present.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a kit for preparing a bioactive composition containing non-crosslinked hyaluronic acid and platelet-rich plasma.

The kit includes a surface-modified centrifuge tube having an inner surface, a modified hyaluronic acid solution contained in the surface-modified centrifuge tube, and a modified separation gel contained in the surface-modified centrifuge tube.

A zeta potential of the inner surface is not greater than −25 mV.

The modified hyaluronic acid solution includes the non-crosslinked hyaluronic acid and a solvent. A pH value of the modified hyaluronic acid solution ranges from 7.0 to 7.8. A zeta potential of the non-crosslinked hyaluronic acid is not greater than −40 mV. The modified hyaluronic acid solution is disposed at an inner bottom of the surface-modified centrifuge tube.

A zeta potential of the modified separation gel is not greater than −25 mV. The modified separation gel is disposed above the modified hyaluronic acid solution.

In certain embodiments, the surface-modified centrifuge tube may be obtained by subjecting an untreated inner surface of a centrifuge tube to a first plasma treatment, so as to convert the centrifuge tube into the surface-modified centrifuge tube having the inner surface derived from the untreated inner surface.

In certain embodiments, the zeta potential of the inner surface may range from −50 mV to −40 mV. In certain embodiments, a zeta potential of the untreated inner surface may range from −20 mV to −10 mV.

In certain embodiments, the centrifuge tube may be made of a material selected from the group consisting of borosilicate glass, soda-lime glass, cesium glass, potassium glass, lithium glass, polycarbonate, polyethylene terephthalate, and polypropylene. According to the present disclosure, the centrifuge tube made from the aforesaid material can exhibit heat resistance and chemical resistance, so as to withstand a plasma treatment.

In an exemplary embodiment, the first plasma treatment is oxygen plasma treatment.

In certain embodiments, the modified hyaluronic acid solution may be obtained by subjecting a hyaluronic acid solution containing an untreated non-crosslinked hyaluronic acid and the solvent to a second plasma treatment, so as to convert the hyaluronic acid solution into the modified hyaluronic acid solution containing the non-crosslinked hyaluronic acid and the solvent. The non-crosslinked hyaluronic acid is formed from the untreated non-crosslinked hyaluronic acid.

In certain embodiments, a zeta potential of the untreated non-crosslinked hyaluronic acid may range from −30 mV to −25 mV.

In certain embodiments, the solvent may be selected from the group consisting of phosphate-buffered saline (PBS), tris(hydroxymethyl)aminomethane (Tris) buffer, 4-(2-hydroxyethyl)-1-piperazineethanesulfonic acid (HEPES) buffer, physiological saline (0.9% sodium chloride (NaCl) solution), lactated Ringer's solution, 2-(N-morpholino) ethanesulfonic acid (MES) buffer, 3-(N-morpholino) propanesulfonic acid (MOPS) buffer, and combinations thereof.

According to the present disclosure, the solvent has an appropriate pH value (7.0 to 7.8) and suitable ionic strength to provide favorable biocompatibility and buffering capacity, thereby stabilizing the molecular structure of the non-crosslinked hyaluronic acid and maintaining platelet activity.

In an exemplary embodiment, the second plasma treatment is oxygen-nitrogen plasma treatment.

In certain embodiments, a weight-average molecular weight of the non-crosslinked hyaluronic acid may range from 100 kDa to 6000 kDa. In certain embodiments, the weight-average molecular weight of the non-crosslinked hyaluronic acid may range from 1500 kDa to 3000 kDa.

In certain embodiments, the non-crosslinked hyaluronic acid may be present in an amount ranging from 1 wt % to 5 wt % based on 100 wt % of the modified hyaluronic acid solution.

In certain embodiments, the modified separation gel is obtained by subjecting a separation gel to a third plasma treatment (or chemical modification), so as to convert the separation gel into the modified separation gel.

In certain embodiments, the zeta potential of the modified separation gel may range from −60 mV to −45 mV. In certain embodiments, a zeta potential of the separation gel may be −10 mV.

In certain embodiments, the separation gel may be made of a material selected from the group consisting of a polysiloxane (e.g., polydimethylsiloxane (PDMS)), a polyester (e.g., polylactic acid (PLA), and polycaprolactone (PCL)), copolymers thereof (e.g., poly(lactic-co-glycolic acid) (PLGA)), poly(methyl methacrylate) (PMMA), a polyacrylate, and polyether ether ketone (PEEK).

In an exemplary embodiment, the third plasma treatment is oxygen plasma treatment.

In certain embodiments, the modified separation gel may have an anionic functional group. In certain embodiments, the anionic functional group may be selected from the group consisting of a carboxylate group, a sulfonate group, a phosphate group, a sulfate group, and combinations thereof.

In certain embodiments, a specific gravity of the modified separation gel may range from 1.040 to 1.085.

The disclosure will be further described by way of the following examples. However, it should be understood that the following examples are solely intended for the purpose of illustration and should not be construed as limiting the disclosure in practice.

### EXAMPLES

### Preparation of Platelet-Rich Plasma (PRP) Separation Kit

### Example 1

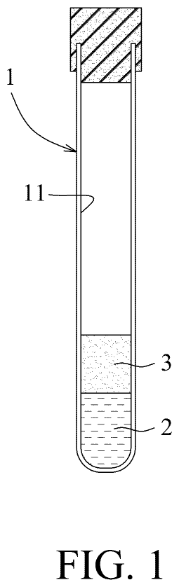

In this example, a method for preparing a kit of the present disclosure was described as follows. Referring to FIG. 1, the kit included a surface-modified centrifuge tube 1, a modified hyaluronic acid solution 2, and a modified separation gel 3.

The surface-modified centrifuge tube 1 was prepared according to the following procedures. First, a centrifuge tube made of borosilicate glass was placed in a reaction chamber, and an untreated inner surface (having a zeta potential approximately ranging from −20 mV to −10 mV) of the centrifuge tube was exposed to ambient air. Subsequently, the untreated inner surface of the centrifuge tube was subjected to oxygen plasma treatment (low-temperature plasma; pressure: 0.5 Torr; power: 50 W; frequency: 13.56 MHz; treatment time period: 60 seconds), so as to convert the centrifuge tube into the surface-modified centrifuge tube 1 having an inner surface 11 formed from the untreated inner surface. A zeta potential of the inner surface 11 ranged from −50 mV to −40 mV.

The modified hyaluronic acid solution 2 was prepared according to the following procedures. First, an untreated non-crosslinked hyaluronic acid (having a weight-average molecular weight of approximately ranging from 1500 kDa to 3000 kDa, and a zeta potential of −28.5 mV±1.2 mV) was dissolved in a 0.9×-1.5× phosphate-buffered saline (PBS; pH 7.4; serving as a solvent), so as to obtain a hyaluronic acid solution with the untreated non-crosslinked hyaluronic acid being present in an amount of 1 wt % based on 100 wt % of the hyaluronic acid solution. Thereafter, the hyaluronic acid solution was placed in a reaction chamber, and then subjected to oxygen-nitrogen plasma treatment with a mixture of 70% oxygen and 30% nitrogen (low-temperature plasma; pressure: 40 mTorr; power: 50 W; frequency: 13.56 MHz; treatment time period: 5 minutes), so as to convert the hyaluronic acid solution into the modified hyaluronic acid solution 2 containing non-crosslinked hyaluronic acid and the 0.9×-1.5× phosphate-buffered saline. The non-crosslinked hyaluronic acid was formed from the untreated non-crosslinked hyaluronic acid, and had a zeta potential of −48.7 mV±1.6 mV.

The modified separation gel 3 was prepared according to the following procedures. First, acrylic acid was added to a polyester-based separation gel (having a zeta potential of −10 mV) until a concentration of the acrylic acid reached 10 g/100 mL, so as to obtain a mixture, and then the mixture was placed into a reaction chamber. Subsequently, the mixture was subjected to oxygen plasma treatment (oxygen flow rate: 50 sccm; pressure: 0.3 Torr; power: 100 W; treatment time period: 60 seconds), so as to convert the mixture into the modified separation gel 3, which had an zeta potential ranging from −60 mV to −45 mV, which contained carboxylate groups, and which had a specific gravity of 1.058.

Afterward, the modified hyaluronic acid solution 2 was disposed at an inner bottom of the surface-modified centrifuge tube 1, and the modified separation gel 3 was disposed above the modified hyaluronic acid solution 2, thereby obtaining the kit for preparing a bioactive composition containing the non-crosslinked hyaluronic acid and platelet-rich plasma of the present disclosure. In other words, the kit, as shown in FIG. 1, included the surface-modified centrifuge tube 1, the modified hyaluronic acid solution 2 contained in the surface-modified centrifuge tube 1, and the modified separation gel 3 contained in the surface-modified centrifuge tube 1. The kit of Example 1 featured an all-in-one pre-filled configuration. That is, the modified hyaluronic acid solution 2 and the modified separation gel 3 were pre-loaded into the surface-modified centrifuge tube 1 prior to use.

### Example 2

The procedures for preparing the kit of Example 2 were generally similar to those of Example 1, except that the untreated non-crosslinked hyaluronic acid in the hyaluronic acid solution was present in an amount of 2 wt % based on 100 wt % of the hyaluronic acid solution.

### COMPARATIVE EXAMPLE

For comparison purpose, a conventional kit serving as the kit of Comparative Example was prepared as follows. In brief, a centrifuge tube made of borosilicate glass was initially provided, and then a polyester-based separation gel (having a zeta potential of −10 mV) was loaded into the centrifuge tube, so as to obtain the conventional kit including the centrifuge tube and the polyester-based separation gel.

### Preparation of Bioactive Composition or Platelet-Rich Plasma (PRP)

### Application Example 1 (AE1)

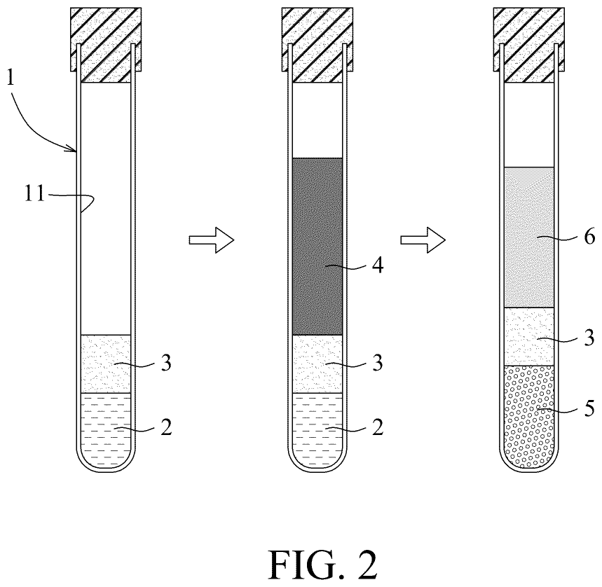

Referring to FIG. 2, first, a blood sample 4, which had a volume of 6 mL to 8 mL and contained an anticoagulant, was loaded into the kit obtained in Example 1, and then subjected to centrifugation at a centrifugal force of 1500 G for 8 minutes, so that red blood cells 5 in the blood sample 4 were separated and driven to flow below the modified separation gel 3. Subsequently, the kit along with the resultant blood sample were shaken for 1 minute to 5 minutes, so as to homogenize a suspension portion (containing platelets, plasma, and the modified hyaluronic acid solution 2) located above the modified separation gel 3, thereby obtaining a bioactive composition 6 of AE1, which included platelet-rich plasma (PRP) and the non-crosslinked hyaluronic acid contained in the modified hyaluronic acid solution 2.

### Comparative Application Example 1 (CAE1)

First, a blood sample, which had a volume of 6 mL to 8 mL and contained an anticoagulant, was loaded into the conventional kit obtained in the Comparative Example, and then subjected to centrifugation at a centrifugal force of 1500 G for 8 minutes, so that red blood cells in the blood sample were separated and driven to flow below the polyester-based separation gel. Subsequently, the conventional kit along with the resultant blood sample were shaken for 1 minute to 5 minutes, so as to homogenize a suspension portion (containing platelets and plasma) located above the polyester-based separation gel. Thereafter, the resultant suspension portion was subjected to two cycles of cryoactivation (12 hours of freezing and 0.5 hours of thawing, per cycle), thereby obtaining PRP of CAE1.

### Comparative Application Example 2 (CAE2)

The procedures for preparing the PRP of CAE2 were generally similar to those of Comparative Application Example 1, except that the resultant suspension portion obtained after homogenization was directly used as the PRP of CAE2 without being subjected to cryoactivation.

### <Bio-Efficacy Evaluation>

### Assays of Growth Factor Contents

Each of the bioactive composition 6 of AE1 and the PRP of CAE1 was subjected to assays of growth factor contents (n=10). The results are shown in Table 1 below.

TABLE 1

Content

| Growth factor | AE1 | CAE1 |
|---|---|---|
| Transforming growth factor-beta 1 | 115 ± 35 | 37.5 ± 12.5 |
| (TGF-β1) (ng/mL) | | |
| Epidermal growth factor | 1000 ± 200 | 350 ± 150 |
| (EGF) (pg/mL) | | |
| Interleukin-8 (IL-8) (pg/mL) | 100 ± 50 | 30 ± 20 |
| Basic fibroblast growth factor | 45 ± 15 | 10 ± 5 |
| (bFGF) (pg/mL) | | |
| Platelet-derived growth factor | 30 ± 10 | 7.5 ± 2.5 |
| (PDGF) (ng/mL) | | |
| Vascular endothelial growth factor | 475 ± 325 | 150 ± 100 |
| (VEGF) (pg/mL) | | |
| Insulin-like growth factor | 125 ± 25 | 65 ± 15 |
| (IGF) (ng/mL) | | |
| Total protein (g/L) | 50 ± 10 | 15 ± 5 |
| Total peptide (g/L) | 2.25 ± 0.75 | 0.75 ± 0.25 |

Referring to Table 1, each of the growth factors in the bioactive composition 6 of AE1 exhibited a significantly higher content than that in the PRP of CAE1.

### Measurement of Cell Proliferation Rate

The human keratinocyte cell line HaCaT and the human chondrocyte cell line C28/I2 were provided. Each of the aforesaid two cell lines was cultivated in a Petri dish containing an appropriate amount of a medium, and then incubated an incubator with culture conditions set at 37° C. and 5% CO₂. Medium change was performed every two to three days. Cell passage was performed when the cultured cells reached 90% of confluence.

Thereafter, the HaCaT cells and the C28/I2 cells obtained above were each divided into 3 groups, including a control group, a comparative example, and an experimental group. Each group of the cells was incubated in a respective well of a 96-well culture plate containing an appropriate amount of a medium at 5×10³ cells/well, followed by cultivation in an incubator with culture conditions set at 37° C., 5% CO₂.

Prior to conducting subsequent treatments (i.e., at the 0th hour), the cell number in each group was calculated using a CellCount™ Cell Counting Kit-8 (CCK-8; Topcells CC02-01). Next, the cells in each comparative example were treated with an appropriate amount of the PRP of CAE2, and the cells in each experimental group were treated with an appropriate amount of the bioactive composition 6 of AE1, while the cells in each control group received no treatment. At the 24^{th} hour and the 72^{nd} hour after conducting the abovementioned treatments, cell number in each group was calculated using the CCK-8. The cell proliferation rate of each group at each of the specified time points (i.e., the 24^{th} hour or the 72^{nd} hour) was calculated using the following Equation:

A=(B/C)×100
where A=cell proliferation rate (%)
   B=cell number of each group at a specified time point
   C=cell number of the control group at the specified time point

The results are shown in Table 2 below.

TABLE 2

| | |
|---|---|
| HaCaT cells | C28/I2 cells |

| | 24^{th} | 72^{nd} | 24^{th} | 72^{nd} |
|---|---|---|---|---|
| Control group | 100% | 100% | 100% | 100% |
| Comparative | 115% | 125% | 110% | 130% |
| group | | | | |
| Experimental | 130% | 160% | 130% | 155% |
| group | | | | |

Referring to Table 2, for each of the HaCaT cells and the C28/I2 cells, the cell proliferation rate of the experimental group significantly increased at the 24^{th} hour after treatment with the bioactive composition 6 of AE1, as compared to the cell proliferation rates of the control group, which received no treatment, and the comparative group, which received the treatment with the PRP of CAE2. A consistent trend was also observed at the 72^{nd} hour. These results demonstrate that the bioactive composition 6 of AE1 is capable of enhancing the cell proliferation rate of each of the HaCaT cells and the C28/I2 cells, and exhibits superior efficacy compared to the PRP of CAE2.

### Evaluation of Injection Treatment

Prior to the start of injection treatment conducted subsequently, patients suffering from knee osteoarthritis (8 individuals, each with a Kellgren-Lawrence Grade of 2 or 3) underwent Visual Analogue Scale (VAS) and Western Ontario and McMaster Universities Arthritis Index (WOMAC) assessments; whereas patients suffering from partial-thickness rotator cuff tears (6 individuals) and adhesive capsulitis (4 individuals) underwent the VAS and Constant-Murley Score assessments.

Afterward, a blood sample of each of the patients was collected, and then subjected to plasma separation using the kit of Example 2 in accordance to the procedures generally similar to those of AE1, so as to obtain a bioactive composition, except that the centrifugation was performed at a centrifugal force ranging from 1200 G to 1500 G for 5 minutes to 10 minutes. Subsequently, each of the patients was subjected to injection treatment with 6 mL of a corresponding one of the bioactive compositions (originating from the same individual), which was administered as a single dose into an affected area thereof. Four weeks after the start of the injection treatment, the patients with the knee osteoarthritis underwent the VAS and the WOMAC assessments again, whereas the patients with the partial-thickness rotator cuff tears and adhesive capsulitis underwent the VAS and the Constant-Murley Score assessments again.

The scores obtained from each assessment were averaged. The results are shown in Table 3 below.

TABLE 3

| | |
|---|---|
| Before injection | Four weeks after |
| treatment | injection treatment |

| | |
|---|---|
| Knee | Average VAS score |

| | | |
|---|---|---|
| osteoarthritis | 6.5 | 2.5 |

Average WOMAC score

| | |
|---|---|
| 56 | 34 |

| | |
|---|---|
| Partial-thickness | Average VAS score |

| | | |
|---|---|---|
| rotator cuff | 7 | 3 |

| | |
|---|---|
| tears/adhesive | Average Constant-Murley Score |

| | | |
|---|---|---|
| capsulitis | 48 | 69 |

Referring to Table 3, the average VAS score of the patients with the knee osteoarthritis, the partial-thickness rotator cuff tears or the adhesive capsulitis significantly decreased after receiving the injection treatment with the bioactive compositions, as compared to the average VAS score thereof before receiving the same; and the average WOMAC score of the patients with the knee osteoarthritis also significantly decreased after receiving the injection treatment with the bioactive compositions, as compared to the average WOMAC score thereof before receiving the same, indicating that the bioactive compositions, each of which was obtained using the kit of Example 2 of the present disclosure, are effective in alleviating joint pain, stiffness, and physical dysfunction. In addition, the average Constant-Murley Score of the patients with the partial-thickness rotator cuff tears or the adhesive capsulitis significantly increased after receiving the injection treatment with the bioactive compositions, as compared to the average Constant-Murley Score thereof before receiving the same, indicating that the bioactive compositions, each of which was obtained using the kit of Example 2 of the present disclosure, are effective in improving joint functional performance.

In summary, by virtue of inclusion of the surface-modified centrifuge tube 1, the modified hyaluronic acid solution 2, and the modified separation gel 3, the kit according to the present disclosure indeed allows rapid preparation of the bioactive composition 6, which included platelet-rich plasma (PRP), the non-crosslinked hyaluronic acid, and high concentrations of various growth factors. Furthermore, the bioactive composition 6 prepared using the kit is capable of enhancing cell proliferation rate, alleviating joint symptoms, and improving joint functional performance, thereby achieving the objectives of the present disclosure.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A kit for preparing a bioactive composition containing non-crosslinked hyaluronic acid and platelet-rich plasma, comprising:
a surface-modified centrifuge tube having an inner surface, a zeta potential of the inner surface being not greater than −25 mV;
a modified hyaluronic acid solution contained in the surface-modified centrifuge tube and including the non-crosslinked hyaluronic acid and a solvent, a pH value of the modified hyaluronic acid solution ranging from 7.0 to 7.8, a zeta potential of the non-crosslinked hyaluronic acid being not greater than −40 mV, the modified hyaluronic acid solution being disposed at an inner bottom of the surface-modified centrifuge tube; and
a modified separation gel contained in the surface-modified centrifuge tube, a zeta potential of the modified separation gel being not greater than −25 mV, the modified separation gel being disposed above the modified hyaluronic acid solution.

2. The kit as claimed in claim 1, wherein the zeta potential of the inner surface ranges from −50 mV to −40 mV.

3. The kit as claimed in claim 1, wherein the zeta potential of the modified separation gel ranges from −60 mV to −45 mV.

4. The kit as claimed in claim 1, wherein the solvent is selected from the group consisting of phosphate-buffered saline, tris(hydroxymethyl)aminomethane (Tris) buffer, 4-(2-hydroxyethyl)-1-piperazineethanesulfonic acid (HEPES) buffer, physiological saline, lactated Ringer's solution, 2-(N-morpholino) ethanesulfonic acid (MES) buffer, 3-(N-morpholino) propanesulfonic acid (MOPS) buffer, and combinations thereof.

5. The kit as claimed in claim 1, wherein a weight-average molecular weight of the non-crosslinked hyaluronic acid ranges from 100 kDa to 6000 kDa.

6. The kit as claimed in claim 1, wherein the non-crosslinked hyaluronic acid is present in an amount ranging from 1 wt % to 5 wt % based on 100 wt % of the modified hyaluronic acid solution.

7. The kit as claimed in claim 1, wherein the modified separation gel has an anionic functional group.

8. The kit as claimed in claim 7, wherein the anionic functional group is selected from the group consisting of a carboxylate group, a sulfonate group, a phosphate group, a sulfate group, and combinations thereof.

9. The kit as claimed in claim 1, wherein a specific gravity of the modified separation gel ranges from 1.040 to 1.085.

10. The kit as claimed in claim 1, wherein the surface-modified centrifuge tube is obtained by subjecting an untreated inner surface of a centrifuge tube to a first plasma treatment, so as to convert the centrifuge tube into the surface-modified centrifuge tube having the inner surface formed from the untreated inner surface.

11. The kit as claimed in claim 10, wherein a zeta potential of the untreated inner surface ranges from −20 mV to −10 mV.

12. The kit as claimed in claim 10, wherein the centrifuge tube is made of a material selected from the group consisting of borosilicate glass, soda-lime glass, cesium glass, potassium glass, lithium glass, polycarbonate, polyethylene terephthalate, and polypropylene.

13. The kit as claimed in claim 10, wherein the first plasma treatment is oxygen plasma treatment.

14. The kit as claimed in claim 1, wherein the modified hyaluronic acid solution is obtained by subjecting a hyaluronic acid solution containing an untreated non-crosslinked hyaluronic acid and the solvent to a second plasma treatment, so as to convert the hyaluronic acid solution into the modified hyaluronic acid solution containing the non-crosslinked hyaluronic acid and the solvent, the non-crosslinked hyaluronic acid being formed from the untreated non-crosslinked hyaluronic acid.

15. The kit as claimed in claim 14, wherein a zeta potential of the untreated non-crosslinked hyaluronic acid ranges from −30 mV to −25 mV.

16. The kit as claimed in claim 14, wherein the second plasma treatment is oxygen-nitrogen plasma treatment.

17. The kit as claimed in claim 1, wherein the modified separation gel is obtained by subjecting a separation gel to a third plasma treatment, so as to convert the separation gel into the modified separation gel.

18. The kit as claimed in claim 17, wherein a zeta potential of the separation gel ranges from −5 mV to −15 mV.

19. The kit as claimed in claim 17, wherein the third plasma treatment is oxygen plasma treatment.
